(19) United States
(12) Patent Application Publication
(10) Pub. No.: US 2022/0022590 A1
(43) Pub. Date: Jan. 27, 2022

(54) Customizable Footwear

(71) Applicant: LaChelle Sanders, Chicago, IL (US)

(72) Inventor: LaChelle Sanders, Chicago, IL (US)

(21) Appl. No.: 17/380,298

(22) Filed: Jul. 20, 2021

Related U.S. Application Data
(60) Provisional application No. 63/055,013, filed on Jul. 22, 2020.

(51) Int. Cl. A43B 3/00; A61B 5/11; A61B 5/00

(52) U.S. Cl. CPC A43B 3/0005; A61B 5/112; A61B 5/0004; A61B 5/1112; A61B 2562/0223; A61B 5/6807; A61B 2562/0247; A61B 2560/0204; A61B 2562/0219; A61B 5/4836

(57) ABSTRACT

This present invention relates to a customizable intelligent footwear. More specifically, the intelligent footwear identifies the needs and requirements of the users and accordingly can self-adjust its various features such as ankle support, arch support, quarter support, and others to enhance comfort and performance of the footwear. The intelligent footwear is integrated with a plurality of sensors and chipsets that collects information regarding user's needs, and based on the collected information adjusts different parameters of the footwear. Additionally, the footwear can be easily paired with a compatible software application to allow the wearers to access information related to their footwear features, step counts, sports related content, walking habits, and other relevant information.

### CROSS-REFERENCE TO RELATED APPLICATION

The present application claims priority to, and the benefit of, U.S. Provisional Application No. 63/055,013, which was filed on Jul. 22, 2020 and is incorporated herein by reference in its entirety.

### FIELD OF THE INVENTION

The present invention relates generally to the field of footwear. More specifically, the present invention relates to a customizable footwear configured to adjust to a user's foot in a variety of ways such as width, ankle support, arch support, firmness and more as per the requirements of the user. The customizable footwear of the present invention comprises a sensor system, a chipset, and a control button attached to a standard shoe, that can be operated using a compatible software application for the customizable shoe. The sensor system can determine the needs and requirements of the user and can send the collected information to the compatible application, or the user can manually use the application to adjust various features of the footwear. Accordingly, the present disclosure makes specific reference thereto. Nonetheless, it is to be appreciated that aspects of the present invention are also equally applicable to other like applications, devices, and methods of manufacture.

### BACKGROUND OF THE INVENTION

By way of background, different individuals have different shoe requirements to address their specific needs. Arch support, ankle support, firmness and softness support, and other feature requirements can be different for every individual. As an example, a shoe specifically designed for walking, running, or playing sports may have a different set of features to support the wearer and provide comfort while wearing the shoes or footwear. In addition, a shoe that provides convenience features, for example self-lacing or temperature control, can address certain needs of a wearer with limited mobility.

Arch support is an important feature that helps distribute pressure on feet, provides support to feet, helps with knee, hip, and back-related alignment issues, treats foot ailments, provides a more stable surface for feet and more. Ankle support provides protection for ankle health by preventing any injuries to the ankle area and helps in relieving foot pain during activities. Also, when an individual injures a foot or ankle while playing sports, conventional shoes cannot be adjusted to fit the user's needs. Firmness or softness may need to be adjusted to suit different requirements of the individuals. The quarter is a part of a shoe's upper, that covers the sides and back of the foot behind the vamp. The heel part of a quarter is often reinforced with sturdy material to better support the rear of the foot. Different sets of features are required to suit the needs of different individuals. Individuals with specific needs and issues, such as arch support for example, typically have to spend extra money to search for the correct shoe. Individuals without insurance are most likely unable to afford expensive molded insoles and/or customized footwear.

Additionally, athletes and other health conscious individuals may desire to track their daily steps, walking habits and other activities. To record daily activities, such as step counts and more, individuals are often required to use a smart band or other similar wearable devices. Also, individuals may be interested in sports-related content, news, updates, and other similar information. Generally, to access such information individuals are required to watch news channels, use sports applications (i.e. apps), and access other similar solutions or media. However, it may be difficult to access news channels or sports applications all the time.

Therefore, there exists a long felt need in the art for an improved shoe that can be customized to suit and fit individuals, as well as fulfill requirements for feedback and other information. There is also a long felt need in the art for a shoe that eliminates the need to buy separate shoes of having different features to accommodate different purposes. Additionally, there is a long felt need in the art for a shoe that allows the users to easily adjust shoe features such as ankle support, arch support, quarters, lace or strap tension, temperature, and more, as per the needs and requirements of the users. Moreover, there is a long felt need in the art for a shoe that enables the users to track their daily steps, walking habits, and other similar features. Further, there is a long felt need in the art for a shoe that obviates the need to wear smart bands or other similar wearable devices to monitor step count or more. Furthermore, there is a long felt need in the art for a shoe that enables the users to easily access content related to sports. Finally, there is a long felt need in the art for an improved shoe that is lightweight and customizable as per the requirements of the users.

The subject matter disclosed and claimed herein, in one embodiment thereof, comprises a smart footwear with integrated sensors. The smart footwear is configured to enable adjustment of ankle support, tightness, firmness, softness, temperature, width, and flexibility, as per the needs of a wearer. The smart footwear further comprises: an upper body configured to cover a foot of the wearer; a sole attached to the bottom of the upper body; the sole having a top surface and a bottom surface; a midsole for housing electronic components; the footwear having a plurality of sensors interwoven in the upper body and the sole for scanning the foot of the wearer and identifying pressure needs of the foot; and, the bottom surface having a push button for adjusting ankle support of the wearer. The footwear having a wireless communication module to connect to a remote electronic device for providing notifications of sensor information, and receiving pressure adjustment instructions for adjustment of the upper body and sole pressure and flexibility.

In this manner, the novel intelligent footwear of the present invention accomplishes all of the forgoing objectives, and provides a relatively comfortable and customizable wearable article that allows the users to customize the shoe features such as ankle support, arch support and other features to suit their needs and requirements. The intelligent shoe of the present invention is also user friendly, as it allows the users to easily track step counts, and other daily habits that may be helpful for athletes or health enthusiasts, and allows them to access content related to sports, and more.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a smart footwear with integrated sensors. The smart footwear is configured to adjust ankle support, tightness, firmness, softness, width and flexibility as per the needs of a wearer. The smart footwear further comprises: an upper body configured to cover a foot of the wearer; a sole attached to the bottom of the upper body; the sole having a top surface and a bottom surface; a midsole for housing electronic components; the footwear having a plurality of sensors interwoven in the upper body and the sole for scanning a foot of the wearer and identifying pressure needs of the foot; and, the bottom surface having a push button for adjusting ankle support of the wearer. The footwear having a wireless communication module to connect to a remote electronic device for providing notifications of sensor information and receiving pressure adjustment instructions for adjustment of the upper body and sole pressure.

In yet a further embodiment of the present invention, a wireless smart footwear with in-shoe sensors and a processor is disclosed. The footwear includes: a plurality of pressure-sensing sensors preferably distributed in a pattern having a shape corresponding to an outline of a human foot; a built-in battery to provide a supply voltage to the sensors; the processor configured to receive information from the pressure-sensing sensors and instructs the pressure pump and magnet to inflate the footwear and customize the pressure; and, the processor is further configured to transmit pressure information to a smartphone application installed in a paired electronic device and receive customization instructions from the smartphone application, which may be voice command capable. The pressure-sensing sensors, and the processor in combination, determines the arch support, ankle support, and quarter support based on the sensed pressure.

In yet a further embodiment of the present invention, a method for providing arch support from footwear based on wearer requirements is described. The method includes: initially pairing a smart footwear with an electronic device using a wireless communication channel; wirelessly receiving an arch support instruction from a software application installed in the electronic device by a processor of the footwear; and inflating the sole of the footwear to provide the required or desired arch support to the wearer.

In yet a further embodiment of the present invention, a method for providing ankle support from footwear based on wearer requirements is described. The method includes: initially pushing a push button positioned at a bottom surface of the sole of the footwear; receiving an instruction by a processor for providing ankle support; and, increasing the firmness or tightness of the ankle support to provide the required ankle support to the wearer.

In yet a further embodiment of the present invention, an intelligent footwear is disclosed. The footwear includes: a pressure sensor for measuring pressure exerted on the foot while wearing the footwear; a temperature sensor for monitoring the wearer's foot temperature; an ultrasonic sensor for sensing proximity or location relative to various objects; an inertial magnetic measurement for performing gait analysis; and, a piezo-electric pedometer for counting the number of steps walked by the wearer. The sensor information can be transmitted to a paired electronic device using a wireless communication channel established using a wireless communication module. A wearer can use the temperature sensor information to activate/deactivate a heating element, pad, or sheet positioned under the insole of the shoe. Additionally, the pressure sensor information can be used to activate a rotatable winder to autonomously or hands-free tighten (i.e. wind) or loosen (i.e. unwind) the laces of the shoe. The winder can be positioned or embedded in the midsole of the shoe.

The intelligent footwear can be manufactured using copper woven technology that aides with swelling, inflammation for everyday workers, individuals with disabilities, and athletes. The footwear helps in determining walking habits, and orthopedic needs of the wearer. The footwear can come in various styles, such as high tops, quarter tops, mid tops, boots, and low tops.

In yet a further embodiment of the present invention, an intelligent footwear with a tag such as a barcode or a QR code is disclosed. The tag is configured to be scanned by a camera allowing an embedded URL to be scanned allowing a user to download a software application and access new content, such as sports updates.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a side view of an intelligent and customizable footwear of the present invention in accordance with the disclosed architecture;

FIG. 2 illustrates a block diagram showing exemplary components of smart and customizable footwear of the present invention in accordance with the disclosed architecture;

FIG. 3 illustrates a block diagram showing exemplary built-in sensors of the customizable footwear of the present invention in accordance with the disclosed architecture;

FIG. 4 illustrates a schematic diagram showing wireless connection of the intelligent and customizable footwear of the present invention with a companion smartphone application installed in an electronic device in accordance with the disclosed architecture;

FIG. 5 illustrates a flow diagram showing exemplary steps for operation of the customizable footwear for adjusting according to the specific needs of a wearer in accordance with the disclosed architecture;

FIG. 6 illustrates a perspective view of intelligent footwear of the present invention connected to the software application in accordance with the disclosed architecture;

FIG. 7 illustrates a perspective view of customizable footwear for adjusting the ankle support according to the specific needs of a wearer in accordance with the disclosed architecture;

FIG. 8 illustrates a perspective view showing use of a push button of the smart footwear of the present invention for adjusting the arch support according to the specific needs of a wearer in accordance with the disclosed architecture;

FIG. 9 illustrates an exemplary user interface of a software application used for transmitting instructions and receiving information from the smart footwear of the present invention in accordance with the disclosed architecture; and

FIG. 10 illustrates a perspective view of customizable footwear of the present invention having a digital tag in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for an improved shoe that can be customized to suit and fit individuals, as well as fulfill requirements for feedback and other information. There is also a long felt need in the art for a shoe that eliminates the need to buy separate shoes of having different features to accommodate different purposes. Additionally, there is a long felt need in the art for a shoe that allows the users to easily adjust shoe features such as ankle support, arch support, quarters, lace or strap tension, temperature, and more, as per the needs and requirements of the users. Moreover, there is a long felt need in the art for a shoe that enables the users to track their daily steps, walking habits and other similar features. Further, there is a long felt need in the art for a shoe that obviates the need to wear smart bands or other similar wearable devices to monitor step count or more. Furthermore, there is a long felt need in the art for a shoe that enables the users to easily access content related to sports. Finally, there is a long felt need in the art for an improved shoe that is lightweight and customizable as per the requirements of the users.

The present invention, in one exemplary embodiment, is a novel wireless smart footwear with built-in shoe sensors and a processor is disclosed. The footwear includes: a plurality of pressure sensing sensors, in one potential embodiment, distributed in a pattern having a shape corresponding to an outline of a human foot; a built-in battery to provide a supply voltage to the sensors and electronic components; the processor configured to receive information from the pressure sensing sensors and to instruct the pressure pump and magnet to inflate the footwear and customize the pressure; and, the processor is further configured to transmit pressure information to a smartphone application installed in a paired electronic device and receive customization instructions from the smartphone application. The pressure-sensing sensors and the processor in combination determines the arch support, ankle support and quarter support, lace or strap tension, based on the sensed pressure.

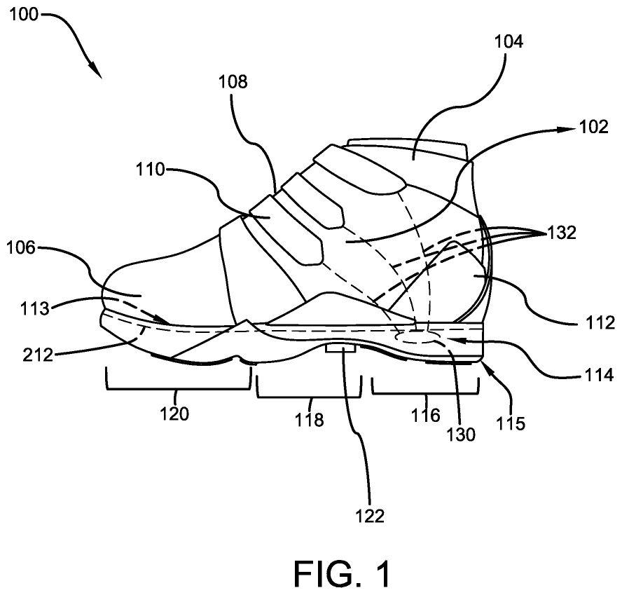

Referring initially to the drawings, FIG. 1 illustrates a side view of an intelligent and customizable footwear of the present invention in accordance with the disclosed architecture. The intelligent and customizable footwear 100 in the present embodiment is in the form of a shoe. The shoe can be one of high tops, quarter tops, mid tops, boots or low tops. The footwear 100 includes an upper body 102, an insole 113, a midsole structure 114, and an outsole 115. A variety of materials can be used for constructing upper body 102, including materials that are conventionally utilized in footwear uppers. The upper body 102 can be formed from one or more portions of leather, synthetic leather, natural or synthetic textiles, polymer sheets, polymer foams, mesh textiles, felts, non-woven polymers, and/or rubber materials, for example. The upper body 102 includes an ankle opening 104 for receiving a foot of a user allowing the upper body 102 to cover and secure the foot. The upper portion 102 includes a vamp area 106 which is the front and center part of a shoe's upper that covers the front of the foot.

The vamp area 106 includes a throat 108 and the throat 108 can include a desired closure mechanism 110 such as laces, straps, hook and loop closures or others. The footwear 100 includes a midsole 114 affixed to the bottom of the upper body 102. The midsole 114 includes a bottom surface or outsole 115 and a top surface. The top surface is covered by the upper body and the foot of a user can be placed on an insole 113 proximal to the top surface of the midsole 114. The bottom surface or outsole 115 includes three portions along the bottom surface, in particular, a heel region 116 configured to be under a heel of a user wearing the footwear 100, a middle arch region 118 configured to be under a foot's arch portion of a user wearing the footwear 100 and a forefoot region 120. The footwear 100 includes a lateral side 112 which is positioned to extend along a lateral side of the foot and generally passes through each of regions 116, 118, 120.

The footwear 100 of the present invention is a smart footwear and is configured to provide comfort to the wearer by customizing tightness, firmness, softness support, width, lace or strap tension, temperature, and flexibility of the footwear 100 based on the requirements of the wearer. A plurality of built-in sensors scans the foot of the wearer and senses the pressure exerted on the sole and the upper body. Based on the scanned information, the footwear 100 can be customized to fit the shape of the foot of the wearer. By way of internal sensors and electronic components, the intelligent footwear 100 of the present invention includes a plurality of sensors, a positioning or GPS chip, a wireless communication chip, a pressure pump, a heating element, a tension winder, a solar panel, a battery, a plurality of LEDs, a microprocessor, a built-in memory and a plurality of additional sensors for the comfort and convenience of the shoe 100 wearer. Various components of the footwear 100 are designed to adjust to the user foot in a variety of ways such as width, flexibility, support, firmness and more. Once the shoe is worn by a wearer, the sensors integrated into the shoe 100 can determine the specific needs of the wearer and can adjust the footwear 100 features accordingly.

The bottom surface or outsole 115 includes a push button 122 in the middle arch region 118, that is configured to adjust the ankle support as per the needs and requirements of the users. The push button 122 on the outsole 115 allows a wearer, such as an athlete, to adjust the firmness or tightness of the ankle support. Additionally, the midsole 114 can house a battery powered tension winder 130 for adjusting the tension of the straps or laces 110 of the shoe 100. The tension winder 130 can rotate (i.e. wind) in one direction to reel tensioning cords 132 in order to increase tension of the straps 110, or can rotate (i.e. unwind) in another direction to unreel tensioning cords 132 in order to decrease tension of the straps 110.

Various components such as sensors, chips, and more can be woven into the material of the footwear 100 or can be applied as a separate layer covering the footwear 100. The sensors can measure the pressure and determine the need for an arch support, or an ankle support, and can adjust the footwear 100 based on the determined needs. Additionally, the sensors can determine the need to raise the sole region 118 and can adjust the sole region 118 of the footwear 100 accordingly.

The footwear 100 can be made of copper woven technology that aides with swelling, inflammation for everyday workers, individuals with disabilities and athletes. The footwear 100 of the present invention is lightweight and can be designed generally in a weight range from about five (5) ounces to about twelve (12) ounces. In one potential embodiment, the footwear 100 weighs eight (8) ounces.

It should be appreciated that the components of the footwear 100 are integrated into each shoe comprising the pair of footwear 100, and in case the components determine that only one foot needs adjustments to its footwear, the components can adjust each shoe individually with the desired changes accordingly.

The sensors can determine various health monitoring parameters, support requirements, environment parameters, and other relevant data, and can transmit the collected information to a paired smartphone application (which may be voice command capable) to provide step counts, walking habits, orthopaedic needs and more.

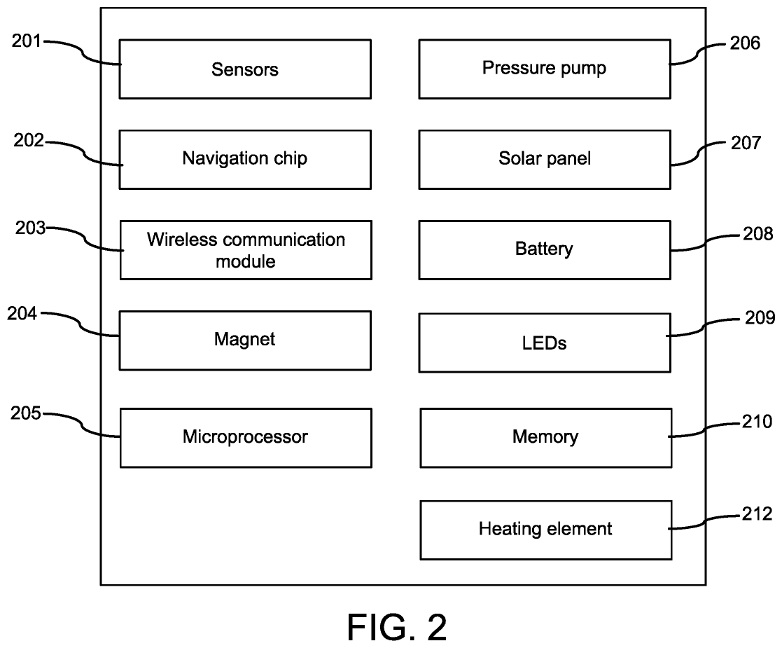

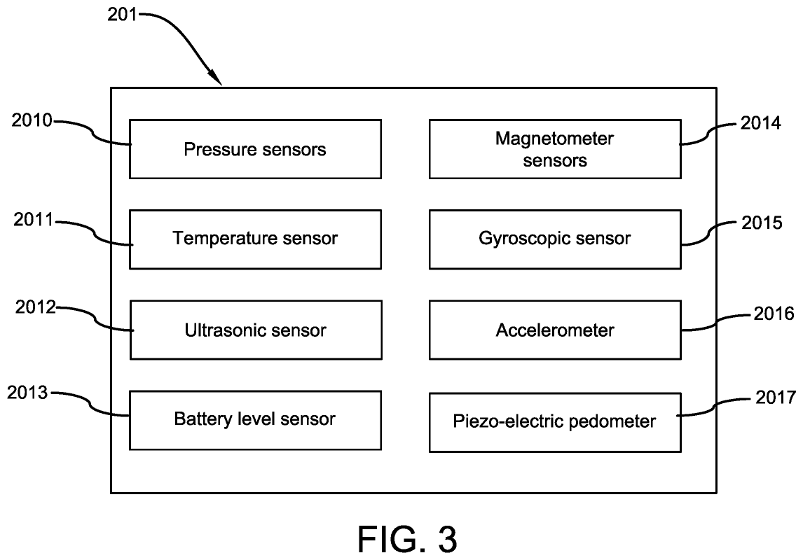

FIG. 2 illustrates a block diagram showing exemplary components of a smart and customizable footwear of the present invention in accordance with the disclosed architecture. The footwear 100 of the present invention includes a plurality of sensors 201 (to be described in more detail in FIG.3), a navigation or GPS chip 202, a wireless communication module 203, a magnet 204, a microprocessor 205, a pressure pump 206, a solar panel 207, a built-in battery 208, a plurality of LEDs 209, a built-in memory 210, and a heating element 212. It should be noted that additional components can be included in the footwear 100 to increase the utility of the footwear 100.

The plurality of sensors 201 determine the foot support requirements, health tracking parameters of the wearer, and enables the footwear 100 to self-adjust the support and cushioning to provide comfort to the foot of the wearer. A navigation chipset 202, such as a GPS chip, senses or tracks real-time location or position information and can provide same to a remote device (i.e. a paired smartphone application). The GPS tracking can be used to provide real-time information of military movements (i.e. solo soldier location or mass advancements, etc.), location of stolen merchandise, route tracking, and navigation. The wireless communication module 203 such as a Wi-Fi chip, Bluetooth module, or other similar modules, can be integrated into the footwear 100 to establish a wireless communication channel with a remote electronic device such as a smartphone. The wireless communication channel can be used for transmitting sensor information to the remote electronic device. It should be noted that the electronic device includes a software application (app) paired to a smart phone. The software application includes a written program to be used on the operating system of the electronic device. The magnet 204 helps in intelligent cushioning to improve comfort and performance of the users while wearing the shoe 100. The microprocessor 205 includes a processor or a controller that receives information from sensors 201, a navigation chip 202, and other components to transmit the sensor information to the remote electronic device. The microprocessor 205 can also instruct the sensors and components to provide cushion and support to the wearer's foot by customizing as per the foot anatomy of the wearer. The pressure pump 206 can be used to inflate the sole to suit the different requirements of the users. The battery 208 can be used for providing power to sensors and components of the footwear and can be recharged using a solar panel 207 or through a USB charging feature. The solar panel 207 can be built-in to the shoe and exposed to the exterior for effective charging of the built-in battery 208. The LEDs 209 can be optionally used to indicate the status of battery 208 charge level of the footwear 100. A memory 210 storage device can be integrated into the shoe and can include a volatile or non-volatile memory. The memory 210 storage device can be used for temporarily storing information collected by various sensors 201. The heating pad, sheet, or element 212 can be activated to increase the temperature to the interior of the shoe 100.

FIG. 3 illustrates a block diagram showing exemplary built-in sensors of the customizable footwear of the present invention in accordance with the disclosed architecture. A plurality of sensors such as one or more pressure sensors 2010, a temperature sensor 2011, an ultrasonic sensor 2012, a battery level sensor 2013, a magnetometer sensor 2014, a gyroscope 2015, an accelerometer 2016 and a piezo-electric sensor (i.e. pedometer) 2017 are integrated in the footwear 100 of the present invention to determine support requirements of foot of a wearer.

The pressure sensor 2010 measures foot pressure and accordingly helps in changing the footwear configuration in order to evenly distribute the pressure over the foot to provide a comfortable footwear wearing experience to the users. The temperature sensor 2011 monitors wearer's foot temperature and can transmit this information to the compatible software application installed in a paired electronic device. The ultrasonic sensor 2012 is used for sensing proximity to various objects and helps in assisting blind individuals who wear the footwear 100 for a safe navigation. The battery level sensor 2013 monitors the status of the built-in battery and accordingly indicates to the user to charge the footwear 100 in case the battery is low. The battery level sensor 2013 can display the battery charge status on an exterior of the footwear and can transmit same to the paired software application. The inertial magnetic measurement units composed of magnetometer 2014, gyroscope 2015 and accelerometer 2016 can be used for gait analysis. The piezo-electric pedometer 2017 is used to count the number of steps walked by a user in a specific period of time. The pedometer 2017 can then share the tracked data, such as steps counted, to the paired software application of the wearer.

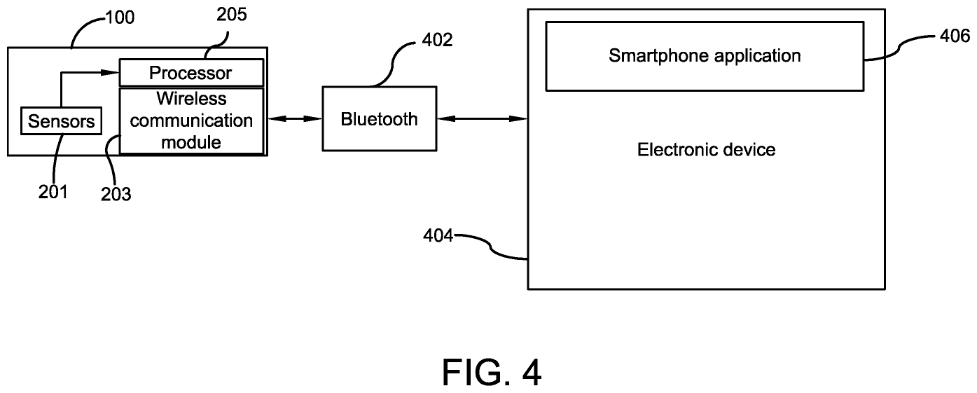

FIG. 4 illustrates a schematic diagram showing the wireless connection of the intelligent and customizable footwear of the present invention with a companion smartphone application installed in an electronic device in accordance with the disclosed architecture. As stated earlier, electronic device 404 can be in the form of a smartphone usable with a special software application 206. The software application 406 includes a written program to be used on the operating system of the smartphone 404 and allows the display screen of the smartphone 404 to display notifications. In a further aspect of the embodiments, the smartphone 404 stores operational software codes that includes an algorithm to process the received information from the footwear 100.

The intelligent footwear 100 connects to the electronic device 404 such as a smartphone, personal digital assistant (PDA), or any other portable device using a wireless communication medium such as low energy Bluetooth 402 using the wireless communication module 203. The plurality of sensors 201 continuously sense the foot of the wearer wearing the shoe and provide information to the microprocessor 205. The microprocessor 205 is programmed to determine specific needs such as arch support needing to be adjusted, or ankle support needing to be adjusted for the wearer and transmits information to the smartphone application 406. The smartphone device 404 having installed companion software application 406 receives real time information related to the arch support needing to be adjusted from the footwear 100 via the Bluetooth 402. The wearer can also provide input using the electronic device to the processor to fit requirements and support while wearing the shoe. For example, the wearer can provide an input to increase arch support by providing an input on the user interface of the software application installed in the electronic device allowing the microprocessor 205 to instruct the sensors to increase the arch support.

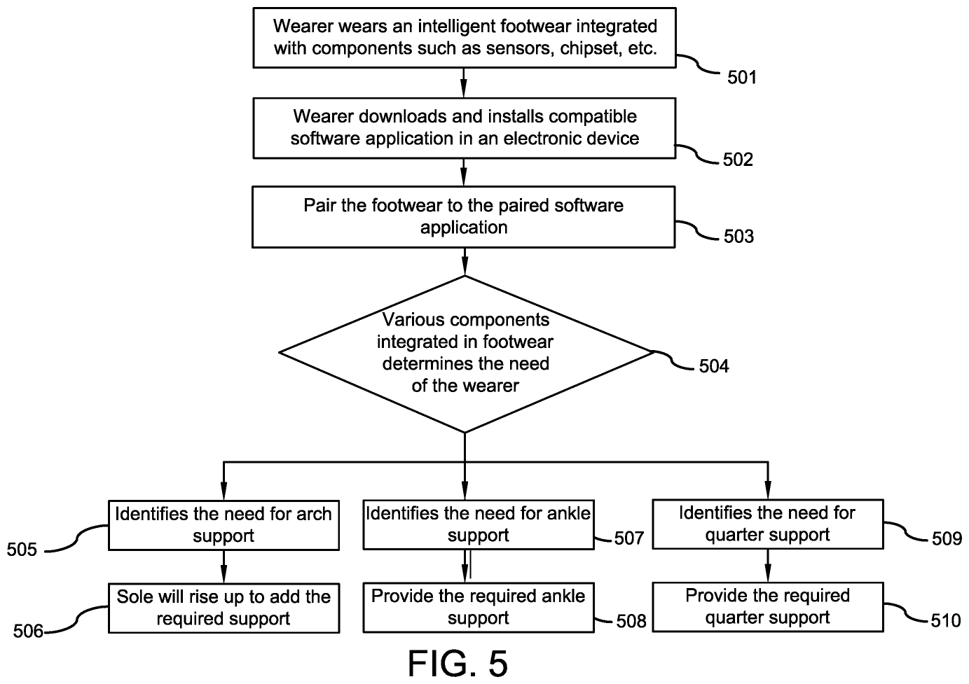

FIG. 5 illustrates a flow diagram showing exemplary steps for operation of the customizable footwear for adjusting according to the specific needs of a wearer in accordance with the disclosed architecture. Initially, intelligent footwear of the present invention designed to adjust width, support, firmness and more of the shoe based on a wearer's requirement is worn by a wearer (Step 501). The user can then install the smartphone application in an electronic device (Step 502). It should be noted that order of the steps 501 and 502 can be reversed in an alternative embodiment. Further, the wearer can download the software application and the application can be used for controlling a plurality of shoes that are owned by the wearer. Each shoe can have a unique identifier allowing the wearer to identify the specific associated shoe. The electronic device, having software application installed, can be paired with the footwear using a wireless communication medium such as Bluetooth (Step 503). The sensors can identify the specific needs of the wearer of the shoe (Step 504). If the footwear 100 determines the need for additional arch support (Step 505), then the built-in sensors and components such as built-in magnets will raise the sole of the footwear to provide the required support (Step 506).

Similarly, when the footwear 100 determines the need for an ankle support (Step 507), the sensors and components can adjust the firmness, lace/strap tension, or tightness to provide the required ankle support (Step 508). Further, when the footwear 100 determines the need for quarter support (Step 509), the sensors and components can adjust the firmness, or tightness to provide the required quarter support (Step 510) to the wearer.

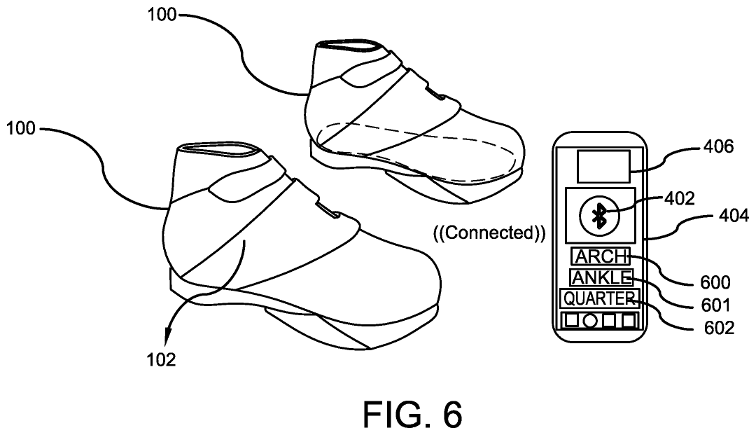

FIG. 6 illustrates a perspective view of intelligent footwear of the present invention connected to the software application in accordance with the disclosed architecture. As shown, software application 406 is installed in an electronic device 404 such as a smartphone, PDA or any other portable device. The improved customizable adjustable footwear 100 connects to the electronic device 404 using a wireless communication medium such as Bluetooth 402. The smartphone application 406 can allow the wearer to adjust the internal structure of the footwear based on the specific needs and comfort level. The smartphone application 406 provides wearers, such as athletes, those that are disabled, and workers on their feet all day, with a plurality of options that allow the application to send an instruction signal to the footwear 100 to customize as per the requirements of the wearer. For example, an arch option 600 allows the wearer to adjust the arch region of the shoes 100 and provides the needed arch support by raising the sole of the footwear 100. Similarly, the smartphone application 406 provides the user with an ankle support 601 option that allows a user to adjust the firmness, lace/strap tension, or tightness of the ankle support. The ankle support 601 inhibits the user from ankle sprains or injuries during exercise, or playing sports such as basketball or volleyball, or other such type of activities. The smartphone application 406 also provides user with a quarter support 602 option that allows user to adjust the quarter part of the shoe's upper body 102. The quarter support 602 option allows the user to adjust the shoes 100 by loosening the quarter panel on the upper body 102 when the user experiences a feeling of tightness and pain while wearing the shoes 100.

It should be noted that when a user provides an input to change a parameter of the footwear on the user interface of the smartphone application 406, a wireless instruction signal is transmitted from the electronic device 404 to the footwear 100. The microprocessor or the integrated chip of the footwear 100 receives the instruction and provides instructions to the sensors of the footwear 100 allowing the footwear 100 to adjust as per the requirements of the wearer.

For each specific adjustment and customization, the software application can provide a plurality of customization options. Also, an instruction can be repeated by the wearer until the required customization and personalization is achieved by the footwear 100. Also, the real-time transmission of instruction signals is performed by the software application to the footwear 100.

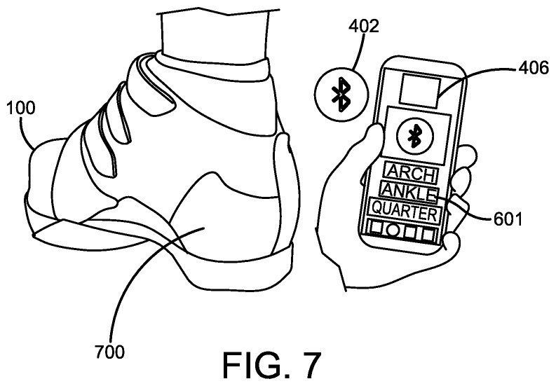

FIG. 7 illustrates a perspective view of customizable footwear for adjusting the ankle support according to specific needs of a wearer in accordance with the disclosed architecture. As shown, the customizable footwear 100 can be connected to the software application 406 using a wireless communication medium such as Bluetooth 402. The smartphone application 406 provides the user with an ankle support 601 option that allows the wearer to adjust the firmness, lace/strap tension, or tightness of the ankle support 700 of the shoe 100. The ankle support 601 inhibits injuries to the user, such as ankle sprains or other injuries during exercise; or, playing sports such as basketball or volleyball, or any other such type of activities. The ankle support 601 provides support needed in the ankle based on the wearer requirements and prevents a user's ankle from becoming painful or swollen because of the walking or standing for extended periods of time.

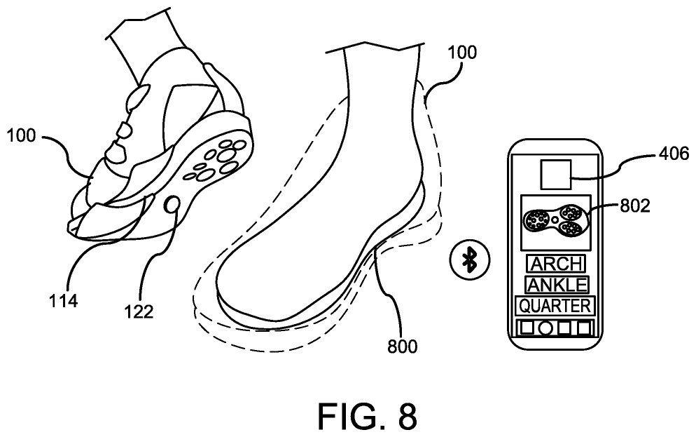

FIG. 8 illustrates a perspective view showing use of push button of the smart footwear of the present invention for adjusting the arch support according to the specific needs of a wearer in accordance with the disclosed architecture. As illustrated, a push button 122 positioned at the bottom surface of the midsole 114 of the customizable footwear 100 allows a wearer such as an athlete to adjust the firmness or tightness of the ankle support. The push button 122 can be manually operated by the wearer. Once the push button 122 is activated, an instruction signal is transmitted to the microprocessor of the footwear for providing the ankle support to the wearer. Simultaneously, the arch 800 of the shoe 100 can be adjusted based on the wearer requirements. When the push button 122 is activated, a user interface of the paired software application 406 also provides a notification 802 showing activation of the push button.

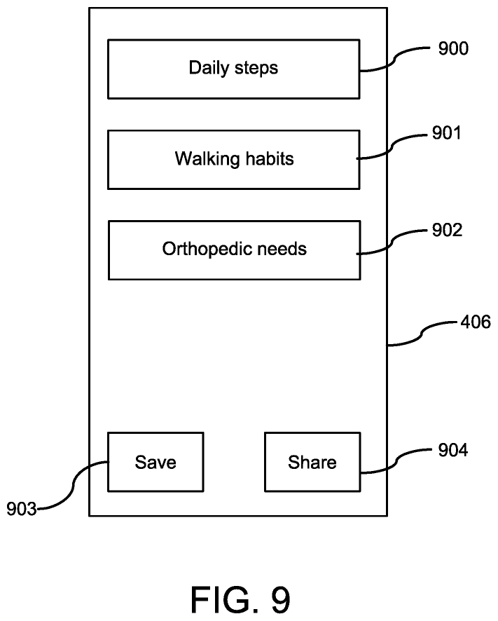

FIG. 9 illustrates an exemplary user interface of the software application used for transmitting instructions and receiving information from the smart footwear of the present invention in accordance with the disclosed architecture. As stated earlier, the sensors of the footwear of the present invention can record daily steps 900, walking habits 901 and the orthopaedic needs 902 of a wearer. This information can be received in real-time or in a specified duration by the software application and can be displayed on the user interface. The daily steps 900 information provides the user with an insight of movement throughout the day. The daily steps 900 can motivate users to keep walking and to establish fitness goals. The walking habits 901, including the weight of the user and the speeds of steps of the wearer throughout the day, can be used to design customized shoes to maximize comfort. The orthopaedic needs 902 based on the walking habits 901 suggest the information related to the stretching and strengthening exercises for the muscles, tendons and joints. The orthopedic needs 902 can also provide the information related to the structure and mechanics of the foot, ankle and leg for individuals who suffer from poor foot mechanics.

The software application 406 can provide the user with an option to save 903 the record of the daily steps 900, walking habits 901 and orthopaedic needs 902 for their future use. The software application 406 can also provide the user with an option to share 904 the record of the daily steps 900, walking habits 901 and orthopaedic needs 902 to other users such as family members, relatives, doctors and more.

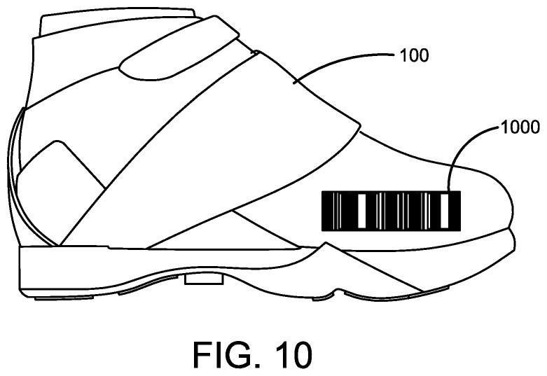

FIG. 10 illustrates a perspective view of customizable footwear of the present invention having a digital tag in accordance with the disclosed architecture. As shown, the customizable footwear 100 includes a tag 1000 in the form of a barcode, QR code or any other scannable code. The tag 1000 can be scanned by a camera of an electronic device to download the companion smartphone application. The tag 1000 contains a hidden URL which, upon being scanned by the camera, takes the user to a weblink from where the user can download the application. In one embodiment, one or more apps can be downloaded by the user on scanning the tag 1000. The user can watch a variety of contents such as sports content, exercise content and any other such type of news content on the downloaded applications.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “intelligent and customizable footwear”, “smart footwear”, “customizable footwear”, “shoe”, and “footwear”, are interchangeable and refer to the intelligent and customizable footwear 100 of the present invention.

Notwithstanding the forgoing, the intelligent and customizable footwear 100 of the present invention can be of any suitable size and configuration as is known in the art without affecting the overall concept of the invention, provided that it accomplishes the above-stated objectives. One of ordinary skill in the art will appreciate that the size, configuration, and material of the intelligent and customizable footwear 100 as shown in the FIGS. are for illustrative purposes only, and that many other sizes and shapes of the intelligent and customizable footwear 100 are well within the scope of the present disclosure. Although the dimensions of the intelligent and customizable footwear 100 are important design parameters for user convenience, the intelligent and customizable footwear 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What includes been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A customizable footwear configured to adjust to a user's foot, the customizable footwear comprising:
an integrated sensor system including at least a temperature sensor, a plurality of pressure sensors, and a battery sensor;
said plurality of pressure sensors sense tightness, ankle support, arch support, and firmness of said footwear relative to said user's foot;
said temperature sensor monitors a temperature of said user's foot;
said battery sensor monitors a status of a built-in battery in said footwear;
said pressure sensors transmit said sensed tightness, said ankle support, said arch support, and said firmness to a paired wireless smartphone application; and
said sensor system further includes at least one control button attached to said footwear to adjust at least one of said tightness, said ankle support, said arch support, and said firmness of said footwear.

2. The customizable footwear of claim 1, wherein said smartphone application sends a first instruction to said at least one control button to adjust at least one of said tightness, said ankle support, said arch support, and said firmness of one said footwear.

3. The customizable footwear of claim 2, wherein said smartphone application sends a second instruction to said at least one control button to adjust at least one of said tightness, said ankle support, said arch support, and said firmness of another said footwear.

4. The customizable footwear of claim 1, wherein said sensor system further includes a magnetometer sensor, a gyroscope, and an accelerometer for measuring a distance traveled.

5. The customizable footwear of claim 1, wherein said sensor system further includes a GPS tracker for tracking in real-time a location of said footwear.

6. The customizable footwear of claim 1, further comprising a solar panel for recharging said battery.

7. The customizable footwear of claim 1, wherein said footwear comprises at least two materials selected from the group consisting of a leather, a synthetic leather, a polymer, a synthetic textile, and a rubber material.

8. The customizable footwear of claim 1, wherein said sensor system further includes a magnetometer sensor, a gyroscope, and an accelerometer for sensing a walker's gait by a wearer of said footwear.

9. The customizable footwear of claim 1, wherein said sensor system further includes a piezo-electric sensor for counting a number of steps walked by a wearer in a specified period of time.

10. A customizable footwear configured to adjust to a user's foot, the customizable footwear comprising:
an integrated sensor system including a plurality of pressure sensors and a battery sensor;
said plurality of pressure sensors sense tightness, ankle support, arch support, and firmness of said footwear relative to said user's foot;
said battery sensor monitors a status of a built-in battery in said footwear;
said pressure sensors transmit said sensed tightness, said ankle support, said arch support, and said firmness to a paired wireless smartphone application;
said sensor system further includes at least one control button attached to said footwear to adjust at least one of said tightness, said ankle support, said arch support, and said firmness of said footwear;
said smartphone application sends a first instruction to said at least one control button to adjust at least one of said tightness, said ankle support, said arch support, and said firmness of one said footwear; and
said smartphone application sends a second instruction to said at least one control button to adjust at least one of said tightness, said ankle support, said arch support, and said firmness of another said footwear.

11. The customizable footwear of claim 10, further comprising a temperature sensor for monitoring a temperature of said user's foot, and
said smartphone application sends a third instruction to said at least one control button to activate a heating element to increase said temperature.

12. The customizable footwear of claim 10, wherein said sensor system further includes a magnetometer sensor, a gyroscope, and an accelerometer for measuring a distance traveled and a proximity.

13. The customizable footwear of claim 10, wherein said sensor system further includes an ultrasonic sensor for measuring a proximity to an object of a wearer of said wearer.

14. The customizable footwear of claim 10, further comprising a solar panel for recharging said battery.

15. The customizable footwear of claim 10, further comprising a winder for tensioning a strap of said footwear; and
said at least one control button activates said winder to rotate in a first direction to increase tension on said strap and to rotate in a second direction to decrease tension on said strap.

16. The customizable footwear of claim 10, wherein said sensor system further includes a magnetometer sensor, a gyroscope, and an accelerometer for sensing a walker's gait by a wearer of said footwear.

17. The customizable footwear of claim 10, wherein said sensor system further includes a piezo-electric sensor for counting a number of steps walked by a wearer in a specified period of time.

18. A method for customizing a fit of footwear to a user's foot, the method comprising:
integrating a sensor system including a plurality of pressure sensors and a battery sensor;
said plurality of pressure sensors sense tightness, ankle support, arch support, and firmness of said footwear relative to said user's foot;
said battery sensor monitors a battery status of a built-in battery in said footwear and displays said battery status on an exterior of said footwear;
adjusting at least one control button attached to said footwear to adjust at least one of said tightness, said ankle support, said arch support, and said firmness of said footwear;
sending a first instruction to said at least one control button to adjust at least one of said tightness, said ankle support, said arch support, and said firmness of one said footwear; and
sending a second instruction to said at least one control button to adjust at least one of said tightness, said ankle support, said arch support, and said firmness of another said footwear.

19. The method of claim 18, further comprising a step of measuring a distance traveled of a wearer of said footwear using a magnetometer sensor, a gyroscope, and an accelerometer.

20. The method of claim 19, further comprising a step of transmitting said sensed tightness, said ankle support, said arch support, and said battery status to a paired wireless smartphone application.
